SEQUENCE LISTING (1 sequence)

SEQ ID NO: 1 - DNA, 50 bp, Artificial Sequence
  Nuclear localisation sequence (NLS)
cacataacgggagggccggcggttaccaggtcgacggatatgacggcagg

(19) United States
(12) Patent Application Publication
(10) Pub. No.: US 2015/0307897 A1
(43) Pub. Date: Oct. 29, 2015

(54) NON-VIRAL VECTOR

(71) Applicant: UNIVERSITY COLLEGE CORK, Cork (IE)

(72) Inventors: Declan Soden, Cork (IE); Gerald O'Sullivan, Cork (IE); Patrick Forde, Cork (IE)

(21) Appl. No.: 14/441,713

(22) PCT Filed: Nov. 7, 2013
(86) PCT No.: PCT/IB2013/059968

§ 371 Date: May 8, 2015

(30) Foreign Application Priority Data
Nov. 8, 2012 (GB) 1220119.0

(51) Int. Cl. C12N 15/85; C07K 14/535; C07K 14/52

(52) U.S. Cl. CPC C12N 15/85; C07K 14/52; C07K 14/535; C07K 14/521; C12N 2800/70; C12N 2830/008

(57) ABSTRACT

The present invention provides a non-viral vector which comprises a sequence encoding an RNA replicase and a nuclear localisation sequence. The vector may also comprise a nucleotide sequence of interest (NOI). The vector may be used to deliver an NOI to a target cell.

### FIELD OF THE INVENTION

The present invention relates to a non-viral vector which may be used for delivery of a nucleic acid sequence.

### BACKGROUND TO THE INVENTION

A vector is a tool that allows or facilitates the transfer of a nucleic acid sequence into a target cell. For a cell to express an exogenous DNA sequence, it must be delivered to the nucleus, whereupon it is transcribed by the host transcriptional machinery. The ability to induce a target cell to express exogenous sequence is an essential tool of biomedical research and offers potential as a therapeutic strategy through gene therapy.

Vectors for genetic delivery may be non-viral or based on a viral system.

Non-viral gene delivery includes plasmids that are introduced into target cells through a variety of transfection methods including electroporation, lipofection, ultrasound and nanoparticle delivery. Each of these transfection strategies aims to facilitate the transportation of the plasmid across the plasma membrane, for example electroporation causes transient disruptions in the integrity of the membrane allowing the plasmid to enter into the cell whilst lipofection packages the plasmid into small lipid particles which are internalised into the cell. In order for the plasmid to be transcribed each must successfully enter the nucleus of a target cell. In practice, however, plasmids enter the nucleus at a very low efficiency, often leading to lower levels of exogenous gene expression than are desired.

Viral-based gene delivery via transduction allows both efficient delivery and expression of exogenous genetic material. The virus life cycle, consisting of gaining entry to a target cell, delivering viral genetic material and hijacking the host biochemical processes to facilitate the expression of viral proteins, is well-suited for manipulation to enable the expression of exogenous genetic material via the insertion of selected nucleic acid sequences into the viral genome. A variety of virus classes have been utilised as genetic vectors, including retroviruses and lentiviruses.

Both retroviruses and lentiviruses contain an RNA-based genome that is converted to DNA by a viral-encoded reverse transcriptase before being integrated into the host genome by an integrase enzyme. Once integrated into the host genome the viral genetic sequence, now termed a pro-virus, is transcribed by the host transcriptional machinery. In addition, because the pro-virus is integrated into the host genome, it is replicated as host genomic sequence and retained in the progeny following cell division. This feature makes the use of retroviruses and lentiviruses favoured in basic research as the prolonged manipulation of gene expression can be achieved. Integration into the host genome can, however, have adverse consequences including integration into genomic sites which may not be permissive of transcription, sites which may disrupt the sequence of essential host genes or sites which lead to transformation of the target cell. This unpredictability of pro-virus integration into the host genome is a particular concern for the use of these viral vectors in gene therapy approaches.

All viral vectors, including retroviruses, lentiviruses and other classes such as adenoviruses, are associated with immunogenic effects when utilised in gene therapy due to their inherent interaction with the host immune system. In addition the use of viral vectors is accompanied by general safety concerns, for example although all viruses must be inactivated before use there is the possibility that the viral vectors may regain replicative capacity, and as such they are general considered more hazardous in comparison to non-viral based vectors.

There is thus a need for alternative vectors for gene delivery which is not associated with the shortcoming of either conventional plasmid vectors or viral vectors.

### DESCRIPTION OF THE FIGURES

FIG. 1 is a schematic diagram comparing the process of mRNA production from the pEEV to a conventional DNA plasmid.

FIG. 2 is a graph showing the relative luciferase levels assayed following expression of a luciferase gene encoded in the pEEV plasmid or a conventional DNA plasmid in a cell-free cytoplasmic extract.

FIG. 3A is a graph demonstrating the relative levels of mRNA derived from a pEEV-encoded lacZ transgene in a variety of murine tissues compared to a conventional pCMV plasmid.

FIG. 3B demonstrates in situ hybridization staining to assess β-Galactosidase protein expression as an assay of pEEV or pCMV-encoded lacZ transgene expression in a variety of porcine tissues.

FIG. 4 is a graph showing the effects of expression of a non-therapeutic lacZ transgene from pEEV on (A) tumour volume and (B) survival in a CT26 tumour model.

FIG. 5 demonstrates TUNEL staining to assess the effect of expression of a non-therapeutic lacZ transgene from pEEV in CT26 tumours.

FIG. 6 is a graph showing the effect of pEEV-GM-CSF/b71 transfection on (A) tumour growth and (B) survival rate in a B16F10 mouse melanoma model.

FIG. 7 is a panel of graphs indicating the effect of pEEV-GM-CSF/b71 transfection in inflammatory cell abundance in both the localised tumour environment and the spleen of a B16F10 mouse melanoma model.

FIG. 8 is a graph showing the effects of pEEV-GM-CSF/b71 transfection on immune memory.

FIG. 9—Therapeutic effect on established solid tumours. Representative CT26 tumour growth curve. Each Balb/C mouse was subcutaneously injected with 1×10⁶ CT26 cells in the flank. When tumours reached an approximate size of 100 mm³ they were treated with pMG (▪), pGT141GmCSF-b7.1 (▴), pEEV (▾) and pEEVGmCSF-b7.1 (♦) or untreated (). 6 mice/groups were used and the experiment was performed twice. Tumour volume was calculated using the formula V=ab²/6. Data is presented as the means±standard error of the mean. It was observed the pEEVGmCSF-b7.1 therapy delayed the growth of the tumours most effectively in comparison to the other groups. 17 days post treatment pEEVGmCSF-b7.1 significantly delayed tumour growth compared to untreated tumour (***P<0.0004) standard therapy vector pGT141GmCSF-b7.1**P<0.002. (B) Representative Kaplan-Meier survival curve of CT26 treated tumours was measured. Only mice treated with pEEVGmCSF-b7.1 survived. 66% of mice survived up to 150 days. All other groups were sacrificed by day 36 (C) Representative growth curve of B16F10 tumour. Each C57BL/6J was subcutaneously injected with 2×10⁵ B16F10 cells in the flank of the mice. When the tumours grew to an approximate size of 100 mm³ they were treated with pMG (▪), pGT141GmCSF-b7.1 (▴), pEEV (▾) and pEEVGmCSF-b7.1 (♦) or untreated (). 6 mice/groups were used and the experiment was performed twice. 12 days post treatment pEEVGmCSF-b7.1 significantly delayed tumour growth compared to untreated tumour (**P<0.0001) standard therapy vector pGT141 GmCSF-b7.1 (*P<0.0001). (D) Representative Kaplan-Meier survival curve of B16F10 showing pEEVGmCSF-b7.1 had 100% survival up to 150 days post treatment with all other groups sacrificed by day 28. Similar results were obtained in two independent experiments.

FIG. 10—Percentage of immune cells in tumour and spleen 72 hours post treatment. FIG. 10a Cells were isolated from CT26 tumours and spleens from treated, untreated or healthy control Balb/C mice. They were analysed by flow cytometry in which 20,000 events were recorded. Data represents the mean percentage of CD19⁺ (B cells), DX5⁺/CD3⁺ (NKT cells), DX5⁺/CD3⁻ (NK cells), CD11c⁺ (DC cells), F4/80⁺ (Macrophage cells), CD4⁺ and CD8⁺ (T cells) positive cells at the time of analysis (48 hours) post treatment. Error bars show SD from between 4 mice. The asterisks (*) indicate significant values of *P<0.05, **P<0.01, ***P<0.001 as determined by one-way ANOVA following Bonferroni's multiple comparison pEEVGmCSF-b7.1 compared to untreated tumour. The asterisks () indicate significance values of *P<0.05, **P<0.01, ***P<0.001 as determined by one-way ANOVA following Bonferroni's multiple comparison of pEEVGmCSF-b7.1 compared to the standard vector pGT141GmCSF-b7.1. Similar results were obtained in two independent experiments.

FIG. 11—Percentage of the respective T cells found locally at the site of the B16F10 tumours treated with pMG, pEEV, pGT141GmCSF-b7.1, and pEEVGmCSF-b7.1 or untreated (a) Represents data obtained for the CD4⁺CD25⁺FoxP3⁺ cells (b) CD4⁺CD25⁻FoxP3⁺ cells (c) CD8⁺FoxP3⁺. Data represents the mean of the respective cells. Error bars show SD from 4 animals. The asterisks (*) indicate significant values of *P<0.05 as determined by one-way ANOVA following Bonferroni's multiple comparison pEEVGmCSF-b7.1 compared to untreated tumour. The asterisks () indicate significance values of *P<0.05 as determined by one-way ANOVA following Bonferroni's multiple comparison of pEEVGmCSF-b7.1 compared to the standard vector pGT141GmCSF-b7.1. Similar results were obtained in two independent experiments.

FIG. 12—Cytokine levels (IFN-γ, IL-10, IL-12 and TNF-α) as measured from tumour and spleens isolated from B16F10 tumour challenged treated, untreated and healthy mice. The error bars represent the mean of 4 individual mice±the SEM. The significance of differences was determined by one-way ANOVA following Bonferroni's multiple comparison (*P<0.05, **P<0.01, ***P<0.001 untreated versus pEEVGmCSF-b7.1 and *P<0.05, **P<0.01, ***P<0.001 pGT141GmCSF-b7.1 versus pEEVGmCSF-b7.1. Similar results were obtained in two independent experiments.

FIG. 13—Cytotoxicity of NK and B cells in tumour and spleens of treated mice. Data represents the mean of the respective cells. Error bars show SD from 4 animals. The asterisks (*) indicate significant values of *P<0.05, **P<0.01, ***P<0.001 as determined by one-way ANOVA following Bonferroni's multiple comparison pEEVGmCSF-b7.1 compared to untreated tumour. The asterisks () indicate significance values of **P<0.01 and ***P<0.001 as determined by one-way ANOVA following Bonferroni's multiple comparison of pEEVGmCSF-b7.1 compared to the untreated groups. Similar results were obtained in two independent experiments.

FIG. 14—Tumour protection, cytotoxicity and immune memory. a. Tumour protection was observed in the pEEVGmCSF-b7.1 treated CT26 mice when challenged (s.c.) with 1×10⁶ tumour cells (n=6/group) in the left flanks. ‘Cured’ and naive mice were challenged with CT26 and 4T1 tumour cells. T-hese mice were observed for tumour development. 100% survival was observed in the CT26 cured mice challenged with CT26. All other groups were sacrificed due to tumour burden by day 25. Similar results were obtained in two independent experiments. b. Augmentation of the in vitro cytolytic activities of the spleen after pEEVGmCSF-b7.1 treatment of CT26 tumours, the specific cytotoxicity was greatest at an effector target ratio of 50:1 after 48 hours incubation. Groups included CT26, 4T1 cells, and Naive and ‘CT26 cured’ splenocytes incubated with CT26 and 4T1 cells respectively. The highest cytotoxicity was observed in the CT26 cells incubated with splenocytes obtained from ‘CT26 cure’ mice treated with pEEVGmCSF-b7.1. The data shown represents one of two separate experiments with similar results (n=6/group). c. Adoptive transfer of lymphocytes of CT26 study. Mice (n=6) received s.c., injections of a mixture of mice receiving CT26 cells and splenocytes either from cured or naive mice, a mixture of 4T1 cells and splenocytes either from cured or naive mice, CT26 cells only or 4T1 cells only. All mice receiving mixtures of CT26 cells and splenocytes either from cured from pEEVGmCSF-b7.1 treatment survived up to 150 days whereas tumours developed in all animals within the other groups. d. Interferon gamma production measured from supernatents obtained from stimulated splenocytes collected from adoptive transfer survivors and naive animals and IFN-γ was measured. High levels of IFN-γ were produced by pEEVGmCSF-b7.1 treated mice. The y-axis represents the concentration of IFN-γ in pg/ml of the supernatant from the stimulated splenocytes. Error bars show SD from 6 animals. e. Tumour protection was observed in the pEEVGmCSF-b7.1 treated B16F10 mice when challenged (s.c.) with 2×10⁵ tumour cells (n=6/group) in the left flanks. ‘Cured’ and naive mice were challenged with B16F10 and Lewis lung tumour cells. These mice were observed for tumour development. 100% survival was observed in the B16F10 cured mice challenged with B16F10. All other groups were sacrificed due to tumour burden by day 28. Similar results were obtained in two independent experiments. f. Augmentation of the in vitro cytolytic activities of the spleen after pEEVGmCSF-b7.1 treatment of B16F10 tumours, the specific cytotoxicity was greatest at an effector target ratio of 50:1 after 48 hours incubation. Groups included B16F10, Lewis lung cells, and Naive and ‘B16F10 cured’ splenocytes incubated with B16F10 and Lewis lung cells respectively. The highest cytotoxicity was observed in the B16F10 cells incubated with splenocytes obtained from ‘B16F10 cure’ mice treated with pEEVGmCSF-b7.1. The data shown represents one of two separate experiments with similar results (n=6/group). g. Adoptive transfer of lymphocytes of B16F10 study. Mice (n=6) received s.c., injections of a mixture of mice receiving B16F10 cells and splenocytes either from cured or naive mice, a mixture of Lewis lung cells and splenocytes either from cured or naive mice, B16F10 cells only or Lewis lung cells only. All mice receiving mixtures of B16F10 cells and splenocytes either from cured from pEEVGmCSF-b7.1 treatment survived up to 150 days whereas tumours developed in all animals within the other groups. h. Interferon gamma production measured from supernatents obtained from stimulated splenocytes collected from adoptive transfer survivors and naive animals and IFN-γ was measured. High levels of IFN-γ were produced by pEEVGmCSF-b7.1 treated mice. The y-axis represents the concentration of IFN-γ in pg/ml of the supernatant from the stimulated splenocytes. Error bars show SD from 6 animals.

### SUMMARY OF ASPECTS OF THE INVENTION

The present inventors have developed an enhanced expression vector (EEV) which is a non-viral vector, such as a plasmid, which comprises a sequence encoding an RNA replicase. The RNA replicase is capable of replicating the transcribed plasmid in the cytoplasm of a transfected cell, resulting in considerably higher levels of expression that a conventional plasmid vector.

The present inventors have found that the level of expression can be further increased through the inclusion of a nuclear targeting sequence in the vector.

Thus, in a first aspect, the present invention provides a non-viral vector which comprises a sequence encoding an RNA replicase and a nuclear localisation sequence (NLS).

The RNA replicase may be a viral RNA replicase, such as one derivable from Semliki Forest virus. The RNA replicase may comprise non-structural proteins 1-4 of Semliki Forest virus.

The NLS may comprise a sequence according to SEQ ID No. 1 or a variant thereof having at least 70% identity.

The vector may also comprise a nucleotide sequence of interest (NOI) which may, for example be a therapeutic gene.

The NOI may encode a protein of interest such as a cytokine, chemokine or antigen.

The NOI may encode GM-CSF and/or b71.

The NOI may be or comprise an miRNA or shRNA.

The vector may also comprise a cell-, site- or tissue-specific promoter.

In a second aspect, the present invention provides method for expressing a NOI in a target cell, which comprises the step of delivering the NOI to the target cell using a vector according to the first aspect of the invention.

Once expressed within the cell the RNA replicase may cause replication of the vector and/or the nucleotide of interest in the cytoplasm of the target cell.

In a third aspect, the present invention provides a method for treating or preventing a disease which comprises the step of administering a vector according to the first aspect of the invention to a subject.

The vector may cause exhaustion, cytolysis or apoptosis of the target cell. This may be due to the RNA replicase over-riding the endogenous cellular machinery for replication causing continued RNA production.

Expression of the NOI in target cells of a subject may down-regulate the production and/or activity of T regulatory cells in the subject.

The disease may be a cancer.

In a fourth aspect, the present invention provides a vector according to the first aspect of the invention for use in treating cancer.

In a fifth aspect, the present invention provides the use of a vector according to the first aspect of the invention in the manufacture of a medicament for use in treating cancer.

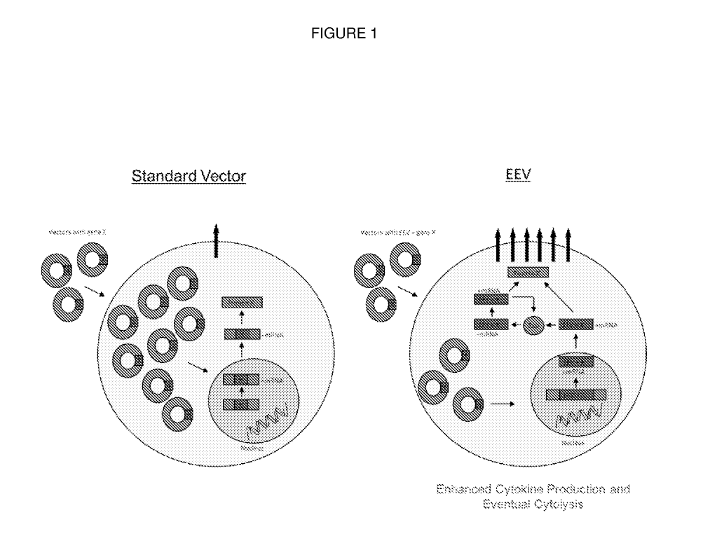

The enhanced efficiency vector described herein thus facilitates high levels of expression from a safe, non-viral vector. The inclusion of an RNA replicase facilitates replicative amplification of vector derived mRNA, meaning that only one copy of the vector must reach the nucleus of the target cell in order to give rise to high levels of transgene expression from the vector (FIG. 1). The presence of a nuclear localisation sequence further enhances expression levels.

### DETAILED DESCRIPTION

Vector

In the first aspect, the present invention provides a vector.

A vector is an agent capable of delivering or maintaining nucleic acid in a host cell. The term includes plasmids, naked nucleic acids, nucleic acids complexed with polypeptide or other molecules and nucleic acids immobilised onto solid phase particles. The vector of the present invention may be a plasmid, in particular a DNA plasmid.

The vector is a non-viral vector, in that it is not based on a virus. It does not include any viral components in order for the vector to gain entry into the cell.

The non-viral vector may comprise a sequence encoding a viral RNA replicase. The viral replicase sequence may be the only viral-derived sequence in the vector.

RNA Replicase

An RNA replicase is an entity, such an enzyme, capable of replicating RNA. An RNA replicase may catalyse the replication of RNA from a single-stranded RNA template. An RNA replicase can also be referred to as an RNA-dependent RNA polymerase.

The replicase may be wholly or partly derivable from a viral RNA replicase.

Viruses with an RNA genome contain or encode an RNA replicase to facilitate genomic replication and are classified based upon the precise nature of the RNA that constitutes their genome. RNA can either be positive-strand RNA (RNA(+)) or negative-strand RNA (RNA(−)). RNA(+) (5′ to 3′) signifies that a particular RNA sequence may be directly translated into protein. Therefore, in RNA(+) viruses, the viral genome can be considered viral mRNA and can be immediately translated by the host cell. RNA(−) (3′ to 5′) is complementary to the required mRNA and thus must be converted to positive-sense RNA by an RNA-dependent RNA polymerase prior to translation. Therefore, like DNA, this RNA cannot be translated into protein directly.

The RNA replicase sequence of the present invention may be derived from non-structural protein (nsp)-1, nsp-2, nsp-3 and nsp-4 of the Semliki Forest Virus (SFV). SFV is an RNA(+) alphavirus with an icosahedral capsid, enveloped by a lipid bilayer derived from the host cell. The RNA(+) genome of SFV contains a 5′ terminal cap, a 3′ terminal poly(A) tail and nine functional proteins which are derived from two open-reading frames. The 5′ two-thirds of the genome are encode polypeptide P1234, from which the nsp-1, nsp-2, nsp-3 and nsp-4 proteins are cleaved, whilst the remaining genome contains structural polypeptides. SFV infects a host cell via receptor-mediated endocytosis followed by membrane fusion stimulated by low-pH, which allows the release of the capsid into the cytoplasm. The liberated capsid is disassembled by ribosomes, resulting in the release of the RNA genome, which is used directly as mRNA to facilitate the synthesis of the non-structural polyprotein (P1234). The polyprotein is autocatalytically processed by the protease activity of nsp-2 to generate the individual components of the SFV RNA replicase, nsp-1, nsp-2, nsp-3 and nsp-4.

The replicative mechanism of the SFV RNA(+) genome consists of a two-step process and occurs in association with specific cytopathic vacuoles. Initially, the RNA(+) template is converted to an RNA(−) intermediary via the action of partly uncleaved polyprotein P123 and free nsp-4. The RNA(−) intermediary acts as a template for the synthesis of multiple copies of the RNA(+) genome, a process that is performed by completely cleaved nsp-1, nsp-2, nsp-3 and nsp-4.

Nuclear Localisation Sequence (NLS)

The vector of the first aspect of the present invention comprises a nuclear localisation sequence (NLS).

An NLS is a nucleic acid sequence that facilitates the transport of a nucleic acid sequence into the nucleus of a target cell.

DNA plasmids utilised in molecular biology and gene therapy are often too large to enter into the nucleus via passive diffusion and therefore require active uptake via proteins such as importin-α, importin-β or transportin. Sequences facilitating the active uptake of DNA into the nucleus are known within the art, an example of which in an enhancer region within the Simian Virus 40 (SV40) viral DNA.

The NLS increases the efficiency with which the vector enters into the nucleus of a target cell.

The inclusion of a nuclear localisation sequence (NLS) enables the vector to gain entry into the nucleus at an efficiency that is far superior to that of a conventional plasmid. This speeds up the process of entry and removes any blockades from the packed cytoplasm that the plasmid must go through in order to gain entry into the nucleus.

The NLS may comprise the sequence shown as SEQ ID 1 or a variant thereof.

SEQ ID No. 1
CACATAACGGGAGGGCCGGCGGTTACCAGGTCGACGGATATGACGGCAGG

Here, the term “variant” means an nucleic acid sequence having a certain identity with the sequence shown as SEQ ID No. 1.

In the present context, a variant sequence is taken to include an NLS which is at least 70, 75, 85 or 90% identical, maybe at least 95 or 98% identical to the sequence shown as SEQ ID No. 1. The variant sequence act as an NLS, i.e. retains the capacity of SEQ ID No. 1 to direct a nucleic acid to the nucleus.

Identity comparisons can be conducted by eye, or more usually, with the aid of readily available sequence comparison programs. These commercially available computer programs can calculate % identity between two or more sequences. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching.

Once the software has produced an optimal alignment, it is possible to calculate % identity. The software typically does this as part of the sequence comparison and generates a numerical result.

Nucleotide Sequence Of Interest (NOI)

The vector of the invention may comprise a nucleotide of interest (NOI).

The NOI may encode a protein of interest (POI).

The NOI may be a DNA or RNA sequence. The NOI may be a whole gene or part of a gene.

The NOI may be a therapeutic or prophylactic gene. The NOI may encode a therapeutic or propylactic protein.

The NOI may be an anti-cancer gene or encode an anti-cancer protein.

A therapeutic gene or protein is expressed within a subject having an existing disease or condition in order to lessen, reduce or improve at least one symptom associated with the disease and/or to slow down, reduce or block the progression of the disease.

A prophylactic gene or protein is expressed within a subject who has not yet contracted the disease and/or who is not showing any symptoms of the disease to prevent or impair the cause of the disease or to reduce or prevent development of at least one symptom associated with the disease.

The NOI may encode a cytokine, chemokine or antigen.

Cytokine

A cytokine is a cell-signalling molecule involved in the generation or maintenance of an immune response.

Examples of cytokines include, but are not limited to, interleukin (IL)-2, IL-4, IL-5, IL-10, IL-12, IL-13, IL-17, IL-25, TNFα, GM-CSF, IFNα, IFNβ and IFNλ.

Granulocyte-macrophage colony stimulating factor (GM-CSF) is a cytokine that known to be secreted by macrophages, T cells, mast cells, NK cells, endothelial cells and fibroblasts. It functions as growth factor stimulating the differentiation of pluripotent hematopoietic stem cells to myeloid stem cells and is required for the development and function of cells throughout the myeloid lineage, including eosinophils, basophils and monocytes.

The vector of the present invention may comprise a nucleic acid sequence encoding for all, or part of, GM-CSF.

Chemokine

A chemokine is a protein associated with the immune system, which is secreted by a cell and is capable of inducing the chemotaxis of cells expressing a receptor recognising the given chemokine.

Example of chemokines include, but are not limited to, CCL1, CCL2, CCL3, CCL4, CCL5, CCL6, CCL7, CCL8, CCL9, CCL10, CXCL1, CXCL2, CXCL3, CXCL4, CXCL5, CXCL6, CXCL7, CXCL8, CXCL9, CXCL10.

b71

b71 is also referred to as CD80. It is a protein found on activated B cells and monocytes that provides a co-stimulatory signal necessary for T cell activation and survival. It is the ligand for two different proteins on the T cell surface: CD28 (for autoregulation and intercellular association) and CTLA-4 (for attenuation of regulation and cellular disassociation).

The vector of the present invention may therefore contain a nucleic acid sequence encoding for all, or part of, the b71 protein.

The vector may comprise a nucleic acid sequence that encodes for GM-CSF and b71.

miRNA/shRNA

The NOI may affect the expression or activity of another molecule, such as a nucleic acid molecule or protein within the target cell. The NOI may, for example be or comprise anti-sense RNA, miRNA or shRNA.

microRNAs (miRNAs) are short non-protein coding RNA molecules (commonly 21-25 nucleotides in length) which are capable of mediating the post-transcriptional regulation of target mRNAs through RNA interference (RNAi) via a mechanism of partially complementary base-pairing. They are generally defined through a natural occurrence in the genome of an organism and generation through a biogenesis pathway involving the actions of the Drosha and Dicer enzyme complexes.

miRNA-mediated RNAi may involve decreasing the level of protein derived from a target mRNA via mechanisms involving either inhibiting or decreasing the efficiency of translation or through direct mRNA degradation. miRNAs may also act to increase the expression of certain proteins.

Short hairpin RNAs (shRNAs) are nucleic acid sequences that generate an RNA molecule containing a hairpin turn and can be used to silence target gene expression via RNAi.

shRNAs are generally distinguished from miRNAs by the use of nucleic acid sequences that differ from those identified within the genome of organisms.

Antigen

The term “antigen” means an entity that is recognised by (i.e. binds specifically) a T-cell receptor and/or antibody.

An antigen may be a complete molecule, or a fragment thereof. The antigen may be, or be derivable from, a naturally occurring molecule.

The vector may act as a vaccine, causing expression of the antigen in vivo which leads to an anti-antigen immune respone.

Where the vector is for use in the treatment of cancer, the nucleic acid sequence may encode all or part of a tumour associated antigen (TAA).

Where the vector is used to treat or prevent an autoimmune disease, the antigen may be an autoantigen. Where the vector is used to treat or prevent an allergic condition, the antigen may be an allergen.

Promoter

A promoter element refers to a sequence of nucleic acids that acts to recruit specific combinations of RNA polymerase, transcription factors and co-factors in order that the transcription of a downstream entity, such as a gene, be co-ordinated and facilitated.

The vector of the invention may comprise a mammalian promoter, so that it is transcribed in a mammalian target cell.

The promoter may be site, tissue or cell-specific. The promoter may be specific for a cancer cell.

Strong promoters include those derived from the genomes of viruses—such as polyoma virus, adenovirus, fowlpox virus, bovine papilloma virus, avian sarcoma virus, cytomegalovirus (CMV), retrovirus and SV40—or from heterologous mammalian promoters—such as the actin promoter or ribosomal protein promoter. Transcription of a gene may be further increased by inserting an enhancer sequence in to the vector. Enhancers are relatively position and orientation independent and be included in the vector at a position 5′ or 3′ to the promoter.

The promoter can additionally include features to ensure or to increase expression in a suitable host. For example, the features can be conserved regions e.g. a TATA box. The promoter may even contain other sequences to affect (such as to maintain, enhance, decrease) the levels of expression of a nucleotide sequence. Suitable other sequences include the Sh1-intron or an ADH intron. Other sequences include inducible elements—such as temperature, chemical, light or stress inducible elements. Also, suitable elements to enhance transcription or translation may be present.

The promoter may, for example, be constitutive or tissue specific.

Examples of constitutive promoters include CMV promoter, RSV promoter, phosphoglycerate kinase (PGK) and thymidine kinase (TK) promoter.

Examples of tissue specific promoters include Synapsin 1, Enolase, α-calcium/calmodulin-dependent protein kinase II and GFAP.

Method of Delivery

In a second aspect, the present invention provides a method for expressing a NOI in a target cell, which comprises the step of delivering the NOI to the target cell using a vector according to the first aspect of the invention.

Once the vector has been transcribed in the target cell the RNA replicase causes replication of the vector in the cytoplasm of the target cell. The RNA replicase causes replication of the NOI in the cytoplasm of the target cell, leading to much greater levels of expression than a conventional plasmid.

The vector may be introduced into target cells using a variety of techniques known in the art, such as electroporation, lipofection or nanoparticle delivery.

Cells may be transfected with the vector in vitro, ex vivo or in vivo.

The RNA replicase and any other NOI are “expressed” in the host cell by being produced as a result of translation, and optionally transcription, of the nucleic acid. Thus the desired expressed products are produced in situ in the cell.

Method of Treatment

In a third aspect, the present invention provides a method for treating or preventing a disease which comprises the step of administering a vector of the present invention to a subject.

The vector may causes expression of the RNA replicase in a target cell in the subject which leads to cytolysis of the target cell because the RNA replicase over-rides the endogenous cellular machinery for replication. Cellular exhaustion may occur from continued RNA production.

The NOI may also comprise a therapeutic or prophylactic gene.

The NOI may down-regulate the production and/or activity of T regulatory cells in the subject.

The disease may be any disease amenable to treatment by selective downregulation or apoptosis of a population of cells, or amenable to treatment by in vivo expression of an NOI or POI. The disease may be an autoimmune disease, allergy or infection. The disease may be a cancer.

The invention will now be further described by way of Examples, which are meant to serve to assist one of ordinary skill in the art in carrying out the invention and are not intended in any way to limit the scope of the invention.

### EXAMPLES

### Example 1

### An Enhanced Expression Vector (EEV) is Capable of Self-Replication Within the Cytoplasm

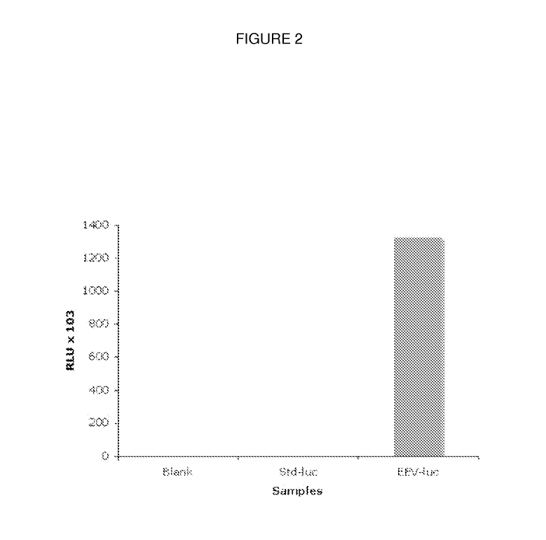

An EEV plasmid containing a luciferase transgene was incubated in a standard rabbit reticulocyte lysate cell-free system (Promega), consisting of cytoplasmic extract free of nuclear material, along with mRNA encoding for T7 RNA polymerase. As standard pCMV plasmid was used as a control and luciferase expression from each plasmid was compared. Only pEEV-luciferase expressed functional protein, as determined by the detection of luminescence, indicating the ability of pEEV but not pCMV to self-express in the presence of a cytoplasmic extract. The present inventors thus demonstrate that a pEEV vector containing an RNA replicase is able to self-replicate and express functional luciferase protein in a cytoplasmic extract free of nuclear material, whilst a conventional plasmid lacks this capacity (FIG. 2).

### Example 2

### EEV Causes Higher Levels of Transgene Expression than a Conventional DNA Plasmids

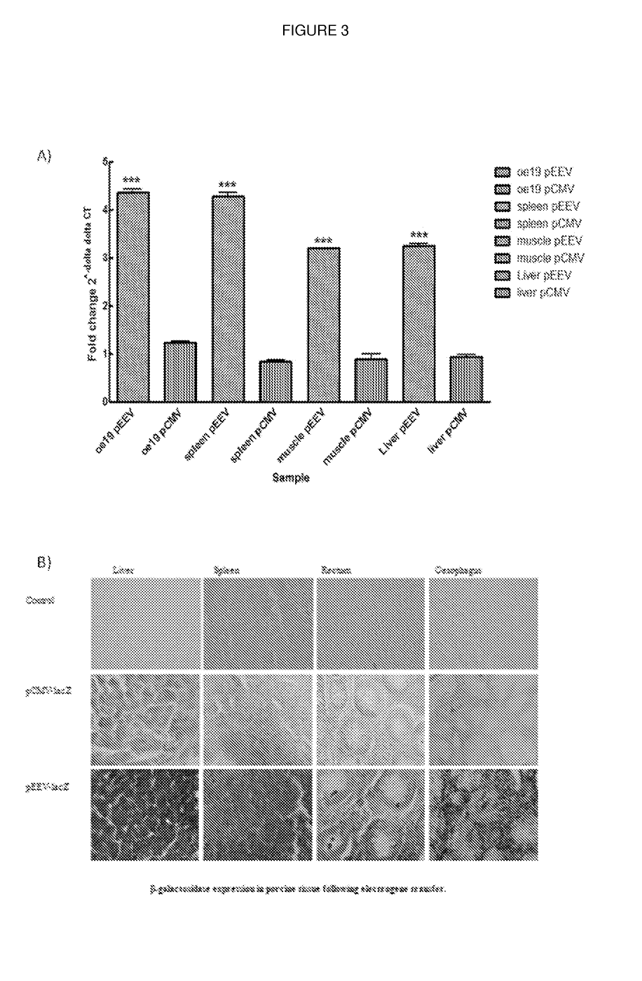

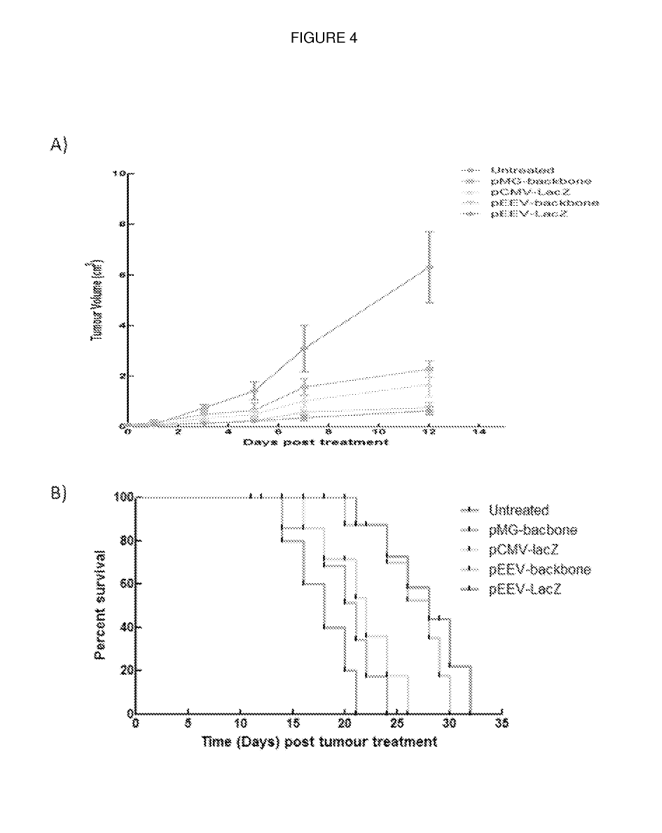

A range of murine tissues, including a subcutaneous tumour (Oe19) and healthy tissue (muscle, liver and spleen), were subject to electroporation-mediated transfection with either 20 ug pEEV or 20 ug conventional pCMV vector, both encoding a lacZ transgene. Tissues were surgically removed after 2 days and qPCR analysis determined that, on average, a four-fold higher level of lacZ transgene expression was derived from the pEEV vector in comparison to the pCMV vector (p<0.0001) across the tissues analysed (FIG. 3A).

The level of pEEV-facilitated transgene expression in a large animal was determined by transfecting a porcine model with either pEEV-lacZ or pCMV-lacZ via electroporation. Transgene expression was determined via examination of β-Galactosidase expression, resulting from the expression of the LacZ transgene. Positive β-Galactosidase staining indicated that the plasmid DNA was successfully delivered and expressed in all tissues analysed. The expression profile of pEEV-lacZ was more abundant in comparison to the standard pCMV plasmid in all tissues analysed, including liver, spleen, rectum and oesophagus. No β-Galactosidase expression was detected in the negative controls (FIG. 3B).

### Example 3

### pEEV-Mediated Expression of a Non-Therapeutic Sequence Results in Cytolytic Activity.

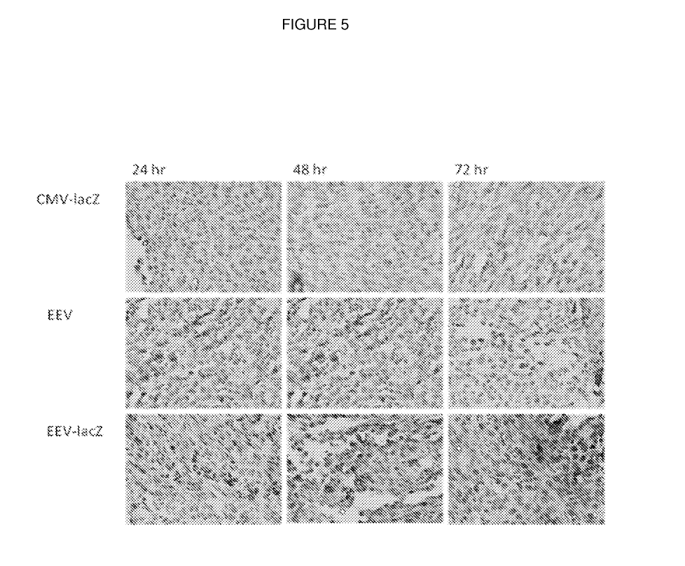

The potential in vivo anti-tumour activity of pEEV was determined in an established tumour model. pEEV-lacZ, pEEV-backbone, pCMV-lacZ and pMG-backbone were transfected into Balb/C mice bearing CT26 tumours via subcutaneous injection with 20 μg of plasmid DNA followed by electroporation. Growth and survival rates were examined and compared to untreated CT26 tumours. The pEEV plasmid bearing a non-therapeutic lacZ transgene significantly reduced tumour volume when compared to the untreated tumour (p<0.05) (FIG. 4A). In addition, improved survival of mice transfected with pEEV was demonstrated (FIG. 4B).

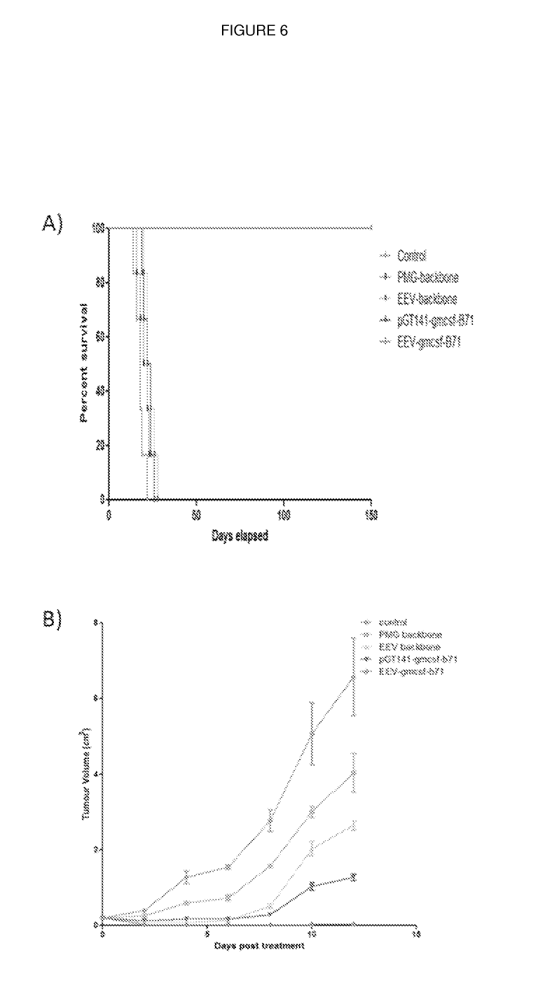

To assess whether the pEEV vector induces apoptotic death in vivo, Balb/C mice bearing CT26 tumours were treated with 20 μg of pEEV, pEEV-lacZ or pCMV-lacZ via subcutaneous injection followed by electroporation. Mice were culled at 24, 48 and 72 hours post treatment and tumours were removed for detecting in situ apoptosis by terminal deoxynucleotidyl transferase-mediated deoxyuridine triphosphate nick-end labelling (TUNEL) staining, which marks apoptotic cells. TUNEL staining demonstrates that the tumours transfected with pEEV-lacZ were abundant in apoptotic nuclei with double-strand DNA breaks which are a hallmark of apoptosis, while apoptosis was not evident in the pCMV treated tumours (FIG. 5). This collective data indicates that improved survival from the non-therapeutic pEEV is due to the oncolytic effect of pEEV.

### Example 4

### pEEV-Mediated Expression of GM-CSF/b71 Confers Enhanced Anti-Tumour Activity

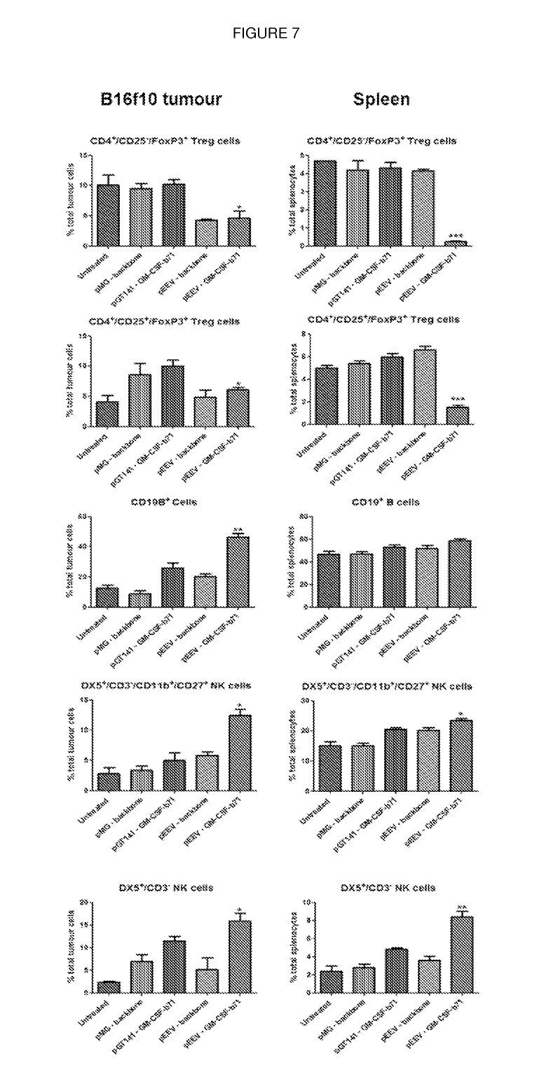

pEEV-GM-CSF/b71 was delivered via electroporation to a B16F10 mouse melanoma model whereupon tumour growth and the survival of the mice post treatment was compared to groups treated with pMG-backbone, pEEV-backbone or pGT141-GM-CSF-b71. Only the mice treated with the pEEV-GM-CSF/b71 were able to eradicate the tumours, as demonstrated by the survival of 100% of mice in this group at 150 days post-treatment (FIG. 6A). All other groups, including those treated with the conventional vectors, were not able to eradicate the tumours (FIG. 6B) and died from tumour burden.

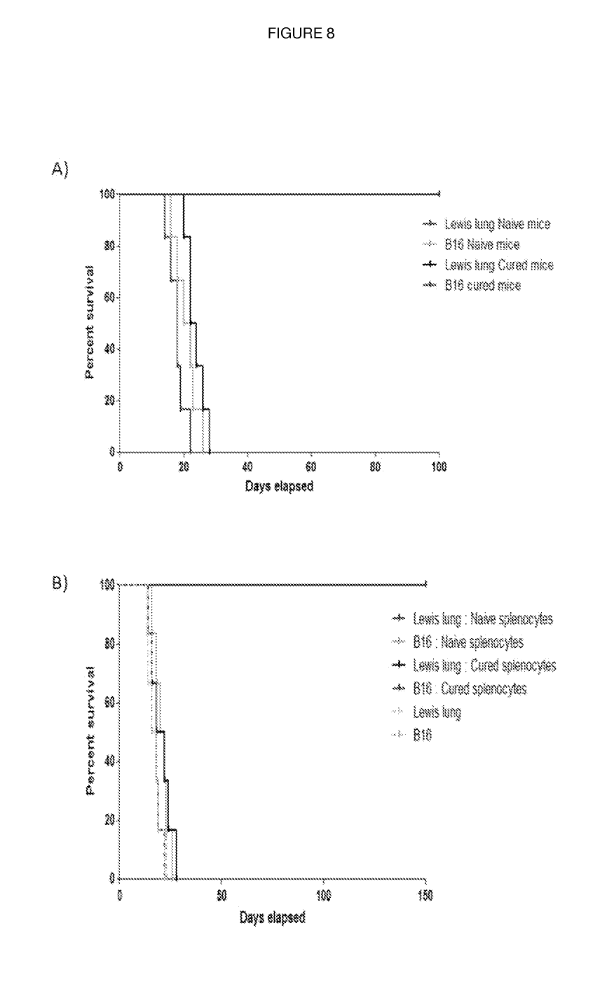

Analysis at seven days post-treatment revealed that pEEV-GM-CSF/b71 had increased production/recruitment of pro-inflammatory cell populations but diminished abundance of T regulatory cells compared to the conventional vector (FIG. 7). Under normal circumstances, T regulatory cell proportions increase as the tumour increases in size and hamper the ability of the host's immune system to eradicate the tumour cells. The pEEV-GM-CSF/b71 therapy resulted in diminution of T regulatory cell populations, both in the local tumour environment and in the spleen, reducing the percentage of T regulatory cells in the tumour from ˜10% in the untreated group to <5% in the pEEV-GM-CSF/b71 group and from 4.5% to <1% in the spleen (FIG. 7). This decrease in the level of T regulatory cells was accompanied by an increase in the recruitment of a number of pro-inflammatory cell types, including innate NK cells and adaptive B cells (FIG. 7).

### Example 5

### Successful Treatment with pEEV-GM-CSF/b71 Leads to Immunological Memory

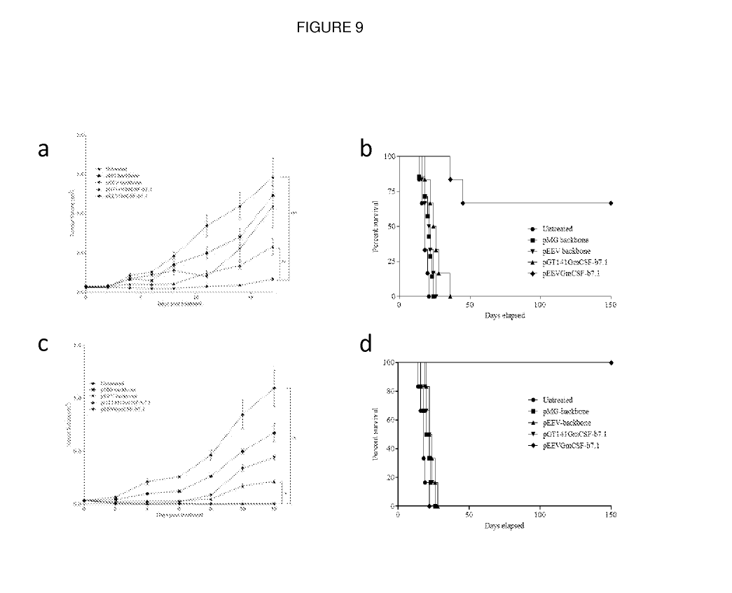

An established B16F10 mouse melanoma model was successfully treated with pEEV-GM-CSF/b71 as described previously (FIG. 8). Following the successful clearance of tumour burden, mice were re-challenged with either the same tumour, B16F10, or a different tumour, in the form of lewis lung cells. 100% of ‘B16 cured’ animals receiving B16F10 survived to Day 100, whilst 0% of lewis lung cells inoculated ‘B16 cured’ mice and 0% of naive mice inoculated with B16 or lewis lung cells survived. This demonstrates an antigen specific immune response to the B16F10 tumour (FIG. 8A).

In a subsequent experiment mice received subcutaneous injections of either B16F10 or lewis lung cells and splenocytes either from ‘B16 cured’ or naive mice. All mice that were challenged with B16F10 and received splenocytes from ‘B16 cured’ mice failed to grow tumours, resulting in survival past the 100 days of the experiment. In contrast, tumours developed in all other groups (FIG. 8B).

### Example 6

### Investigating the Therapeutic Efficacy of pEEVGmCSF-b7.1

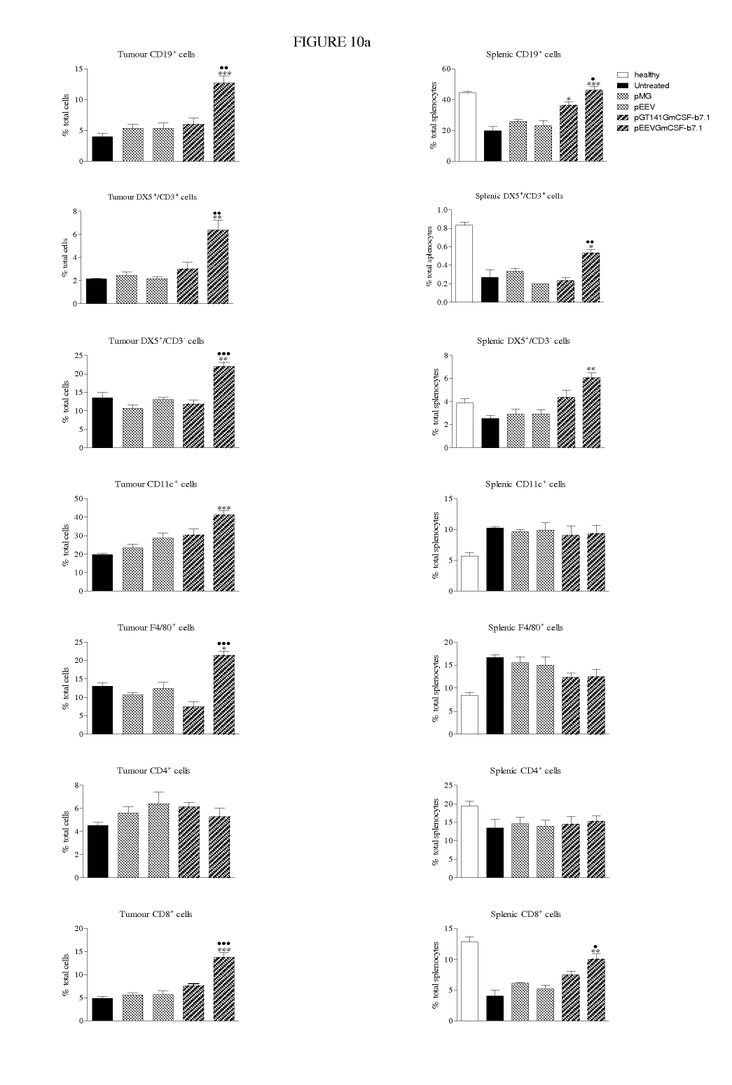

The purpose of this study was to investigate the therapeutic efficacy of pEEVGmCSF-b7.1 and its comparison to a standard vector also expressing GmCSF-b7.1. To test the therapeutic efficacy two tumour types were treated once by electroporating the tumours with the respective plasmids. A CT26 murine colorectal tumour and B16F10 a metastatic melanoma tumour were treated (FIG. 9) with pMG (standard plasmid backbone), pGT141GmCSF-b7.1 (standard plasmid therapy), pEEV (backbone) and pEEVGmCSF-b7.1. The CT26 tumour volumes of all non-electroporated (Untreated) tumours increased exponentially (FIG. 9a). Tumours treated with the empty plasmids, pMG and pEEV also increased in size. The pEEV empty plasmid did however retard the growth of the CT26 tumour during days 8-12 but then rapidly grew exponentially. Both therapeutic plasmids delayed the growth rate of the CT26 tumour. The pEEVGmCSF-b7.1 treated tumours significantly inhibited growth compared to pGT141GmCSF-b7.1 treated tumours (P<0.002) and from the control untreated tumours (P<0.0004). By day 26 post treatment the untreated, pMG and pEEV treated groups were sacrificed due to tumour size (FIG. 9b). While the standard therapy pGT141GmCSF-b7.1 did inhibit tumour growth all from this group were culled by day 36. One mouse was removed on days 36 and 45 from the pEEVGmCSF-b7.1 treated group due to ethical size and the remaining 66% survived. The surviving mice remained tumour free until they were sacrificed on day 150 post treatment for subsequent immune analysis. The B16F10 melanoma cell line was chosen for its aggressive nature to further test the efficacy of pEEVGmCSF-b7.1 therapy. Again the experiment was set up with groups treated with pMG, pGT141GmCSF-b7.1, pEEV and pEEVGmCSF-b7.1 (FIG. 9c). The tumour growth was similar to the CT26 growth profile with untreated tumours growth exponentially with the first untreated tumour sacrifice due to tumour size 12 days post treatment. The pEEVGmCSF-b7.1 delayed the growth compared to the untreated group (P<0.0001) and pGT141GmCSF-b7.1 (P<0.0001). The pMG, pEEV, untreated and pGT141GmCSF-b7.1 treated groups were sacrificed by day 28 (FIG. 9d). Interestingly the standard pGT141GmCSF-b7.1 therapy had no real effect on survival with all mice removed by day 26. The pEEVGmCSF-b7.1 therapy showing a very striking response with 100% survival and all animals remained tumour free for 150 days post treatment until they were removed for subsequent immune analysis.

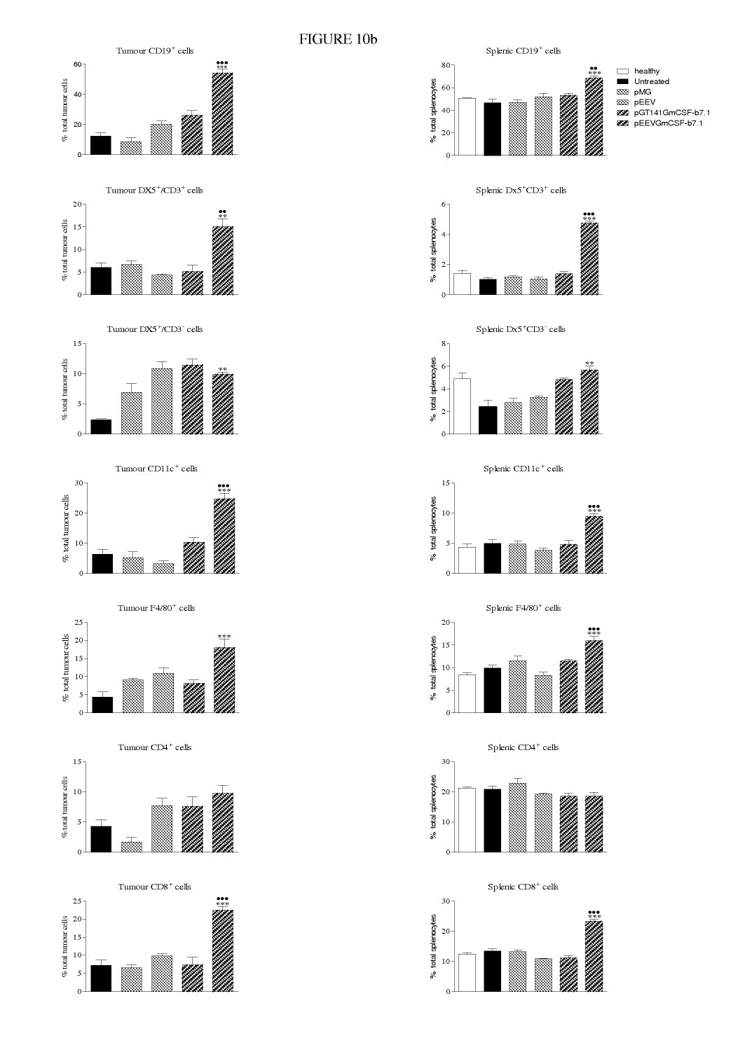

To determine what immune cells were recruited post treatment spleens and tumours were removed and analysed by flow cytometry. A snap shot at 72 hour post treatment was chosen as an analysis time point. Overall there was a global immune response observed with both an innate and adaptive immunity involvement. As shown in FIG. 10a, the CT26 tumour challenged mice showed that CD19⁺, DX5⁺/CD3⁺, DX5⁺/CD3⁻ and CD8⁺ cells were all significantly unregulated in the spleens of pEEVGmCSF-b7.1 treated mice. All cells examined with the exception of CD4⁺ cells were unregulated in the tumours of the pEEVGmCSF-b7.1 treated mice compared to the untreated tumours. It was also observed that pEEVGmCSF-b7.1 treated mice had significantly more splenic CD19⁺ cells (P<0.05) than the standard pGT141GmCSF-b7.1 treated mice. Locally at the tumour, pEEVGmCSF-b7.1 treated mice had significantly more CD19⁺ (P<0.01) DX5⁺/CD3⁻ (P<0.001) F4/80 (P<0.001) and CD8⁺ (P<0.001) cells compared to the pGT141GmCSF-b7.1 treated tumours. There was no real trend for the spleen and tumour CD4⁺ cells with all groups having similar numbers of cells. Spleens from healthy mice were also used as a comparison with CD19⁺, DX5⁺/CD3⁻, CD11c, and F4/80 cells expressed higher in the treated mice than in the healthy mice indicating an involvement of both innate and adaptive immunity. FIG. 10b presents the B16F10 treated tumour immune data and follows a similar trend as observed for the CT26 immune data. CD19⁺ (P<0.001), DX5⁺/CD3⁺ (P<0.01), DX5⁺/CD3⁻ (P<0.01), CD11c⁺ (P<0.001), F4/80 (P<0.001) and CD8⁺ (P<0.001) cells were all significantly higher for the pEEVGmCSF-b7.1 treated mice than the untreated B16F10 tumour. In the spleens of the same animals CD19⁺ (P<0.001), DX5⁺/CD3⁻ (P<0.001), DX5⁺/CD3⁻ (P<0.01), CD11c⁺ (P<0.001), F4/80 (P<0.001) and CD8⁺ (P<0.001) cells again were all significantly present in the pEEVGmCSF-b7.1 treated mice than the untreated mice. When the standard therapy pGT141GmCSF-b7.1 was compared to the pEEVGmCSF-b7.1 CD19⁺ (P<0.001), DX5⁺/CD3⁺ (P<0.01), CD11c⁺ (P<0.001) and CD8⁺ (P<0.001) cells were all significantly recruited indicating pEEVGmCSF-b7.1 recruits a more superior immune recruitment locally at the tumour site. The spleen data had a similar trend as the tumour data with CD19⁺ (P<0.01), DX5⁺/CD3⁺ (P<0.001), CD11c⁺ (P<0.001), F4/80 (P<0.001) and CD8⁺ (P<0.001) cells all up regulated in the pEEVGmCSF-b7.1 treated mice when compared to the standard therapy. CD19⁺, DX5⁺/CD3⁺, CD11c⁺, F4/80⁺ and CD8⁺ cells were all higher in the pEEVGmCSF-b7.1 treated mice than the health mice spleens again indicating the involvement of an innate and adaptive immune response to the treatment.

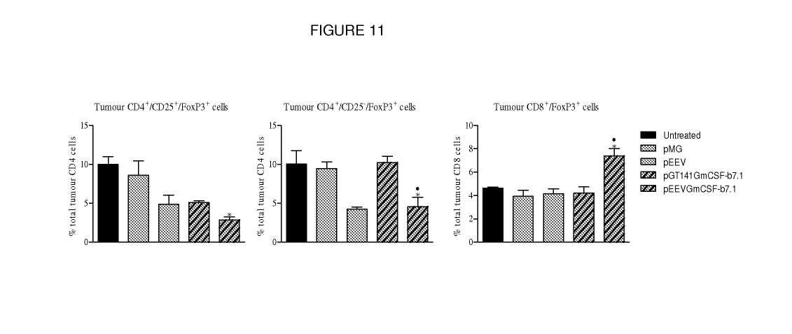

Suppressor T cells are well recognised as a blockade for any cancer therapy and for this reason their presence locally was analysed in the B16F10 tumours of the treated animals (FIG. 11). The pEEVGmCSF-b7.1 treatment group CD4⁺CD25⁺FoxP3⁺ cell population was reduced significantly (P<0.01) compared to the untreated group (FIG. 11a). The CD4⁺CD25FoxP3⁺ tumour cells were also reduced in the pEEVGmCSF-b7.1 treated animals compared to the untreated tumours (P<0.01) whereas there was no change in the standard treatment. CD8⁺CD25⁺ T effector cell population was significantly increased in the pEEVGmCSF-b7.1 treated tumours in comparison to the untreated tumour and the standard pGT141 GmCSF-b7.1 treatment (P<0.01). The pGT141GmCSF-b7.1 treated tumour T effector cell levels were similar to the untreated and backbone.

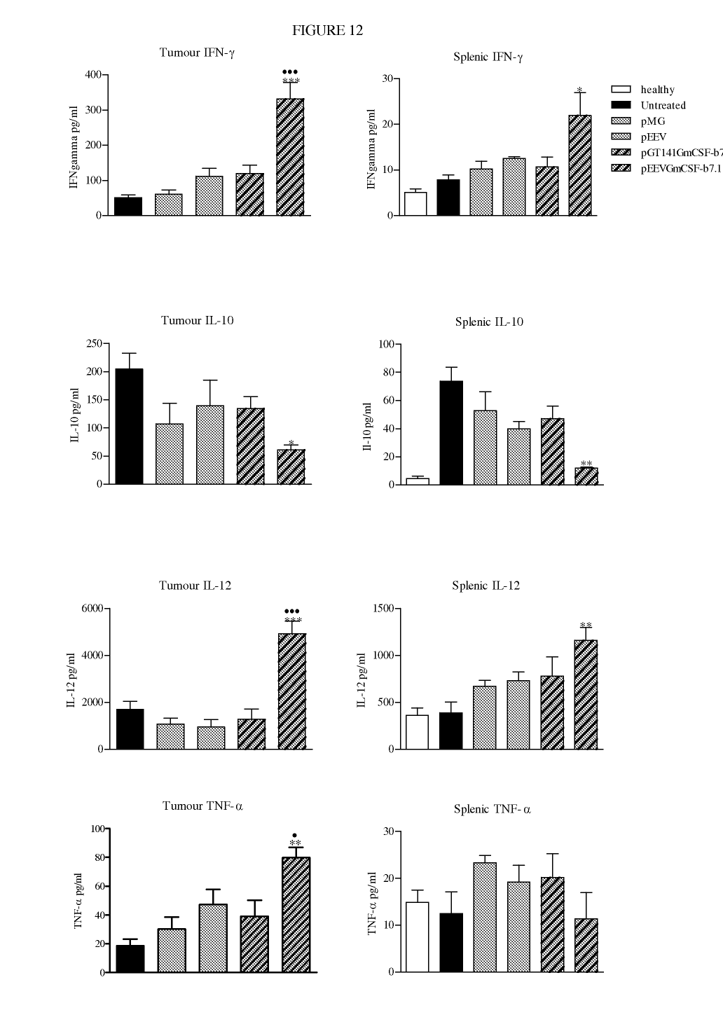

The concentrations of tumour and spleen cytokines in treated; untreated and healthy animals 72 hours post treatment were then examined. Results are presented in FIG. 12. Tumour concentrations of IFN-γ, IL-12 and IL-10 were all elevated for the pEEVGmCSF-b7.1 treatments compared to untreated (P<0.001), pGT141GmCSF-b7.1 (P<0.001) and all the other groups analysed. TNF-α was also elevated for the pEEVGmCSF-b7.1 treatments compared to untreated (P<0.01), pGT141GmCSF-b7.1 (P<0.05) and all the other groups analysed. In contrast the IL-10 levels were lower in the pEEVGmCSF-b7.1 when compared to the untreated group (P<0.05) and all other groups analysed. The spleen data had similar results with elevated levels of IFN-γ, IL-12, IL-10 and decreased levels for IL-10.

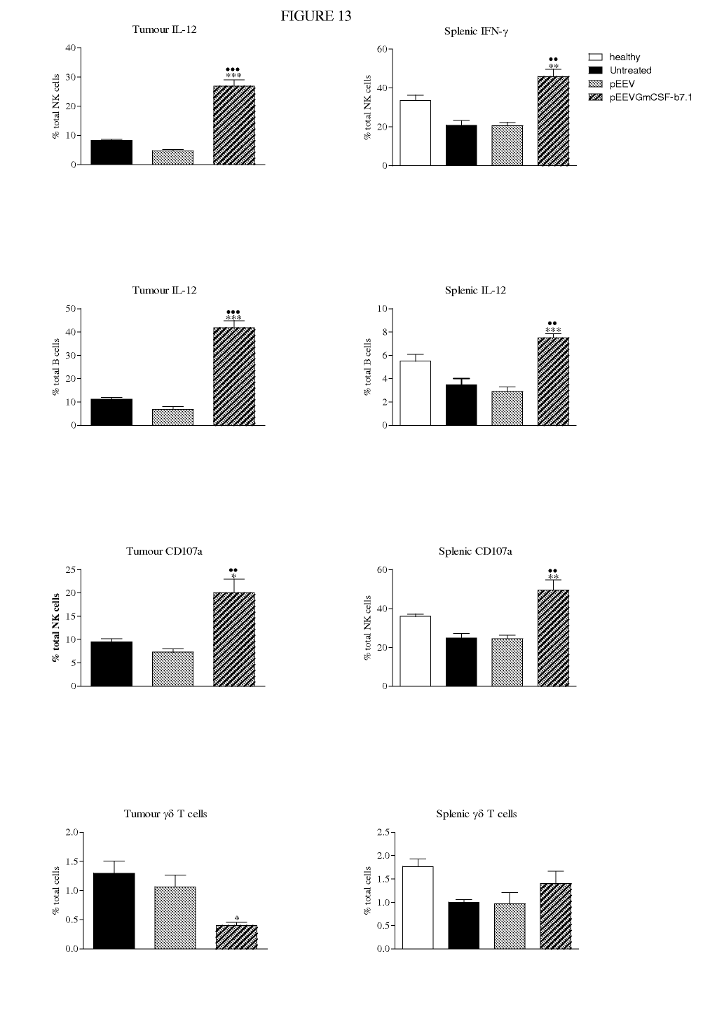

Expression of NK and B cells were significantly elevated in both tumour types of the pEEVGmCSF-b7.1 mice. The cytoxicity/activation capability of NK and B cells in the B16F10 challenged mice was then analysed (FIG. 13). NK cells positive for IFN-γ and CD107a (degranulation marker) were significantly higher in the pEEVGmCSF-b7.1 treated groups compared to the untreated (P<0.001) and the pEEV control (P<0.001) tumours. The splenic data had similar results with elevated levels of IFN-γ positive NK cells compared to the untreated and healthy mice. B cells positive for IL-12 were also significantly higher in the pEEVGmCSF-b7.1 treated groups.

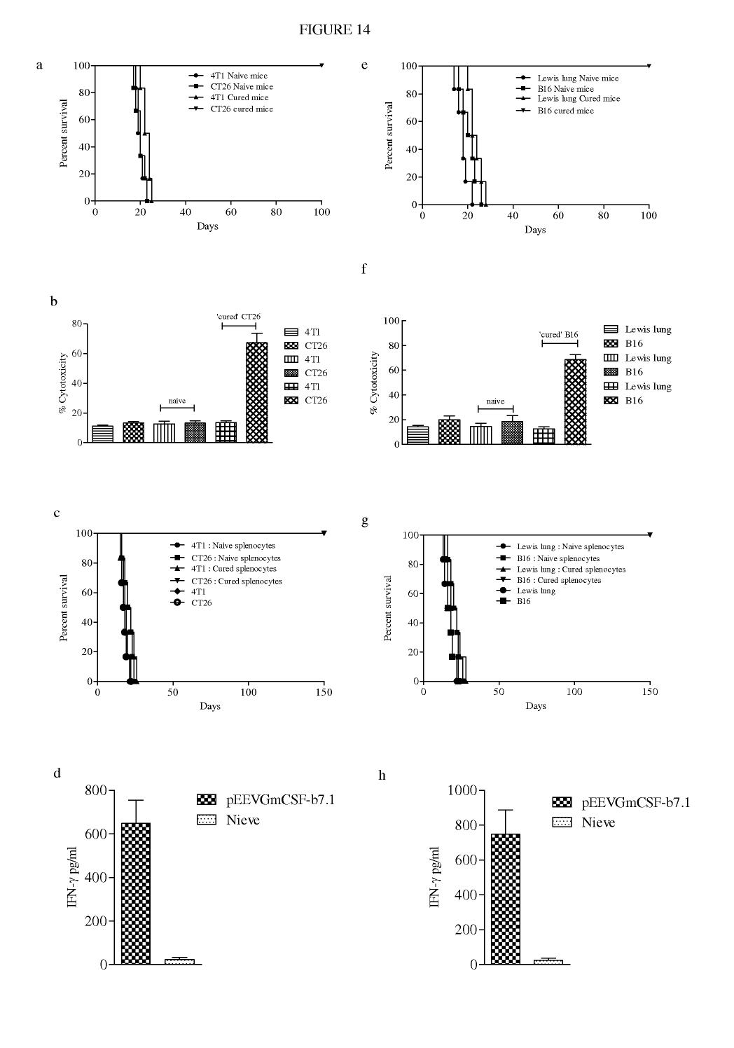

After successful treatment with pEEVGmCSF-b7.1, both CT26 and B16F10 cured mice and naive mice were challenged to determine tumour protection. To compare tumour growth of ‘cured mice’, naive mice of same age were inoculated with the same dose of viable tumour cells (FIGS. 14a and e). To determine tumour specific protection a different tumour (4T1 or Lewis lung) was selected and cured and naive mice were challenged with. Long term tumour-specific protection was seen in the pEEVGmCSF-b7.1 treated ‘cured’ mice group both for the CT26 and B16F10 models, surviving 100 days post challenge. All naive mice succumbed to disease demonstrating that there were protective immune responses in the pEEVGmCSF-b7.1 group where zero mice developed tumours. The immune response was antigen specific, as tumour protection was limited to the CT26 or B16F10 and not to the previously unexposed tumours such as 4T1 and Lewis lung cancer in the respective models. This data suggests that the pEEVGmCSF-b7.1 treatment results in a durable response.

The in vitro cytotoxicity of splenic T lymphocytes against CT26 and B16F10 cells was then determined. (FIGS. 14b and f). The splenic T lymphocytes against CT26 and B16F10 cells were significantly greater in the pEEVGmCSF-b7.1 treated ‘cured’ mice than in the naive mice. To determine the specificity of the cytotoxicity the unexposed tumours 4T1 and Lewis lung were included for the respective model. The splenic T lymphocytes against the 4T1 and B16F10 demonstrated low % cytotoxicity. These results correspond with the observed immunity in vivo (FIGS. 14a and e).

The possible development of an immune mediated anti-tumour activity following pEEVGmCSF-b7.1 was further tested by a modified Winn assay (adoptive transfer), where groups received subcutaneous inoculation of a mixture of CT26 or B16F10 cells and splenocytes from pEEVGmCSF-b7.1 treated ‘cured’ mice or naive mice, a mixture of 4T1 or Lewis lung cells and splenocytes from pEEVGmCSF-b7.1 treated ‘cured’ mice or naive mice, 4T1 or Lewis lung and CT26 or B16F10 in their respective model (FIGS. 14c and g). All mice inoculated with splenocytes from naive mice developed tumours. Mice inoculated with mixtures of splenocytes and 4T1 or Lewis lung developed tumours, whereas no tumour growth was observed in mice inoculated with splenocytes from pEEVGmCSF-b7.1 treated ‘cured’ mice in both the CT26 and B16F10 models indicating the protective effect was antigen specific as observed in the in vitro analysis. Control groups which were inoculated with CT26, B16F10, 4T1 or Lewis lung cells all developed tumours and indicated that the tumours were growing in the correct manner. The tumour protective effect in the mice inoculated with splenocytes from pEEVGmCSF-b7.1 treated ‘cured’ mice in both the CT26 and B16F10 models resulted in prolonged survival (150 days). This suggests adoptive transfer to naive mice of specific antitumour immune response provided protection to tumour challenge.

High levels of IFN-γ were observed from the animals who received the adoptively transferred mixtures of both the CT26 and B16F10 and splenocytes from the pEEVGmCSF-b7.1 treated ‘cured’ mice of the respective model and naive mice of the same age (FIGS. 14 and h). Significantly higher levels of IFN-γ were observed in the adoptively transferred mice in comparison to the naive mice. This observation indicated a high level of Th-1 T cell stimulation in the treatment group and supports the cellular nature of the immune resposes observed in the cytotoxic T lymphocyte assays.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A non-viral vector which comprises a sequence encoding an RNA replicase and a nuclear localisation sequence (NLS).

2. A vector according to claim 1, wherein the RNA replicase comprises a viral RNA replicase.

3. A vector according to claim 2, wherein the RNA replicase is derivable from Semliki Forest virus.

4. A vector according to claim 3, wherein the sequence encoding the RNA replicase comprises non-structural proteins 1-4 of Semliki Forest virus.

5. A vector according to claim 1, wherein the NLS comprises sequence according to SEQ ID NO: 1 or a variant thereof having at least 70% identity.

6. A vector according to claim 1 which also comprises a nucleotide sequence of interest (NOI).

7. A vector according to claim 6, wherein the NOI comprises a therapeutic gene.

8. A vector according to claim 6, wherein the NOI encodes a cytokine.

9. A vector according to claim 8, wherein the NOI encodes GM-CSF.

10. A vector according to claim 6, which encodes a chemokine.

11. A vector according to claim 6, wherein the NOI comprises an miRNA or shRNA.

12. A vector according to claim 6, wherein the NOI encodes all or part of an antigen.

13. A vector according to claim 1 which also comprises a cell- or tissue-specific promoter.

14. A method for expressing a NOI in a target cell, which comprises the step of delivering the NOI to the target cell using a vector according to claim 6.

15. A method according to claim 14, wherein the RNA replicase causes replication of nucleotide of interest in the cytoplasm of the target cell.

16. A method for treating or preventing a disease which comprises the step of administering a vector according to claim 1 to a subject.

17. A method according to claim 16, wherein the vector causes expression of the RNA replicase in a target cell in the subject which leads to cytolysis of the target cell because the RNA replicase over-rides the endogenous cellular machinery for replication.

18. A method according to claim 16, wherein the vector also comprises a nucleotide sequence of interest (NOI), and wherein expression of the NOI down-regulates the production and/or activity of T regulatory cells.

19. A method according to claim 16, wherein the disease is a cancer.

20.-21. (canceled)
